(19) United States
(12) Patent Application Publication
(10) Pub. No.: US 2001/0044658 A1
(43) Pub. Date: Nov. 22, 2001

(54) Method of fastening external prostheses

(76) Inventors: Stephane Auguste, Quetigny (FR); Anne Valentin, Dijon (FR)

Correspondence Address: CROWELL & MORING LLP, Intellectual Property Group, P.O. Box 14300, Washington, DC, 20005-4300, US

(21) Appl. No.: 09/789,002

(22) Filed: Feb. 21, 2001

(30) Foreign Application Priority Data
Feb. 21, 2000 (FR) 00 02 125

(51) Int. Cl. A61F 2/52

(52) U.S. Cl. 623/7

(57) ABSTRACT

It is characterized in that at least one cohesive and elastic pressure-sensitive adhesive patch which can be repositioned on the skin is used, and said patch can be detached from the prosthesis by pulling parallel to the surface of the prosthesis.

Application particularly to the fastening of breast prosthesis.

The present invention relates to an adhesive fastening method for medical devices or prostheses applied to the skin, especially breast prostheses.

### PRIOR ART

The invention relates to the field of medical devices, especially external prostheses, which have to be reversibly and repeatedly fastened to the skin. To take, for example, the case of breast prostheses, women who have undergone ablation of a breast sometimes use, for aesthetic purposes, an external prosthesis which has to be held in place reliably while providing the possibility of being removed overnight and repositioned each morning. Among the various solutions for holding the prosthesis in place, it is possible to use, in addition to the brassiere, a system for direct fastening to the skin. This solution is liked as it takes the weight of the prosthesis, which is placed in the brassiere, off the shoulders and allows, for example, a swimming costume to be worn. In this case, the fastening method may, for example, use a system of the Velcro type called “scraped mesh”, formed by the interaction of a mesh with hooks, attached to the prosthesis, and a mesh with loops, attached to an adhesive support tape bonded to the skin. However, this type of fastening has the drawback of frequently causing irritation to the skin and it is not always tolerated by patients. Another practical method involves a prosthesis whose inner face is permanently covered with an adhesive which, after cleaning the external adhesive face, can be reused. Such adhesive prostheses are described, for example, in document FR 2 757 043, which teaches a fastening by means of patches of silicone gel. Such a self-adhesive prosthesis can thus be used for six months to one year, but, since the adhesive is attached to the prosthesis, the whole assembly must be thrown away when the adhesion becomes too low, with the conblock that the replacement cost is relatively high. It should also be noted that the use of the same adhesive system over a long period requires meticulous cleaning so that the whole assembly remains completely clean, guaranteeing good hygiene. Other, adhesive means are known which are provided for fastening hygiene articles to the skin, for example nappies or sanitary towels, as described in documents WO 98/28015 and U.S. Pat. No. 5 863 977. These adhesives, designed for fastening disposable one-time-use articles, cannot be separated from the object to be fastened and are removed with the said article after it has been used just once. Such adhesives have no need to be repositionable on the skin and separable from the article without destroying the latter.

### SUBJECT OF THE INVENTION

The invention relates to a novel temporary adhesive fastening method for medical devices applied directly to the skin, especially a breast prosthesis, which makes use of patches of a biocompatible adhesive which can be repositioned on the skin and detached from the medical device.

### DESCRIPTION

According to the invention, the method of fastening the medical device to the skin is characterized in that it uses patches cut from a thick sheet of a cohesive and elastic adhesive which can be repositioned on the skin and detached non-reversibly from the said device by pulling and elongating the said sheet of adhesive, parallel to the surface of the said device.

The invention also relates to an external prosthesis that can be fastened to the skin by means of patches of adhesive, the adhesive being able to be repositioned on the skin and able to be separated from the prosthesis by pulling and elongating the sheet of adhesive, in a direction parallel to the surface of the said prosthesis.

For example, the prostheses is a breast prosthesis to which the patches of adhesive are fastened via one of their faces, the other face then being released from its non-stick protective layer in order to be applied against the skin so as to hold the prosthesis in place.

### DETAILED DESCRIPTION

The method according to the invention consists in fastening the prosthesis to the skin by means of at least one patch of pressure-sensitive adhesive having simultaneously the following characteristics:

it is non-allergenic and non-irritating to the skin;

it can be repositioned on the skin;

it has less adhesion to the skin than to the material of which the surface of the prosthesis is made;

its cohesive force is greater than its adhesion to the prosthesis, so that the adhesive patch can be separated from the prosthesis by pulling it in a direction parallel to the adhesive-coated surface, and leaves no traces of adhesive on the surface of the prosthesis (the strip of adhesive can be “stripped off”).

In general, breast prostheses consist of a mass of silicone gel modelled to the shape of the breast and held in an envelope made of polyurethane film. Within the context of the present invention, the inner face of the prosthesis is covered, essentially around the periphery, with one or preferably more patches of adhesive, the thickness of which is sufficient to ensure a cohesive failure force greater than the force of separating it from the prosthesis. According to the invention, a hydrophobic adhesive comprising a blend of synthetic elastomers, a resin and a plasticizer is used. Similar adhesives are described in document U.S. Pat. No. 4,286,077, but these have to be modified in order to meet the conditions of use. Given the particular application, the constituents of the adhesive must be perfectly tolerated by the skin, especially as the adhesive remains in prolonged contact with particularly sensitized skin.

Of the synthetic elastomers used for making a hot-melt adhesive, the preferred ones are triblock elastomers consisting of a saturated central elastomeric block and two thermoplastic styrene-type blocks. These triblock elastomers of A-B-A type, may also contain A-B diblock components in a relatively large amount. Depending on the type of saturated central block, the elastomers may be SEBS elastomers (with a central ethylene-butylene block) or SEPS elastomers (with an ethylene-propylene central block), the average molar mass of which may be between 40 000 and 250 000. Such elastomers suitable for making the adhesive sheet needed for carrying out the invention are sold, for example, by Shell under the name Kraton G, or by Kuraray under the name Septon. Each of these companies provides various grades (according to the styrene content or the degree of polymerization, for example) and it may be advantageous to blend several grades of elastomers in order to optimize the performance of the adhesive; for example, it is possible to blend an SEBS having a high molecular mass such as Kraton G 1651, with an SEBS having a low molecular mass such as Kraton 1726×(which also contains SEB diblock copolymers) in order for the adhesive mass to have both good cohesion and high flexibility. The proportion of elastomer in the adhesive is about to 5 to 35%, preferably between 8 and 25%, expressed by weight.

A preferred type of formulation uses a blend of elastomers of the SEBS or SEPS triblock type which consists of about 1 to 4%, preferably 1 to 2%, of SEBS or SEPS elastomers having a high molecular weight (greater than 180 000) and 7 to 20%, preferably 12 to 20% of SEBS or SEPS elastomers having a medium molecular weight (less than 180 000).

The adhesive also includes a tackifying resin which must allow the said adhesive to exhibit sufficient adhesion to hold the prosthesis in place, while being gentle on the skin. Resins compatible with the central block of the triblock elastomer are chosen, these resins being commonly used in the medical field for the manufacture of bandaging, and of these it is preferred to use resins based on cyclized aliphatic hydrocarbons, and more particularly resins of this type having a melting point above 17° C. Of the commercial products suitable for formulating the adhesive, mention may be made of the resins of the trademark Escorez 5000 sold by Exxon. The proportion of tackifying resin in the adhesive is about 30 to 70% and preferably 55 to 65%.

The adhesive also includes a plasticizer whose function is to give the adhesive sheet flexibility and conformability. Oils or greases well tolerated by the skin and compatible with the elastomer and the resin are used, especially liquid paraffins such as, for example, those sold by Shell under the trademark Ondina. The plasticizer is present in the adhesive in an amount of about 5 to 50%, preferably 10 to 30% and even more preferably from 15 to 25%.

As is customary in the adhesive field, antioxidants, optical stabilizers, dyes and antibacterial agents may also be added to the adhesive to these ensure that the properties of the adhesive sheet are preserved under the conditions of use.

According to one of the preferred embodiments of the invention, it is recommended, for fastening a prosthesis to the skin, to use a sheet of thermoplastic pressure-sensitive adhesive characterized in that it comprises:

1 to 4%, preferably 1 to 2%, of an ABA triblock copolymer having a saturated central block and a molecular weight greater than 180 000;

7 to 20%, preferably 12 to 20%, of an ABA triblock copolymer having a saturated central block and a molecular weight of less than 180 000, the said copolymer possibly containing AB diblock components;

10 to 30% of an oily plasticizer;

55 to 65% of a saturated aliphatic resin whose melting point is above 70° C.;

0.2 to 4% of additives chosen from antioxidants, stabilizers, dyes and antibacterial agents, the said sheet having a thickness of between 0.6 and 3 mm, sufficient for the cohesive force of the sheet to be greater than the force to remove it from a substrate when the adhesive sheet is pulled parallel to the substrate.

Preferably, the sheet of adhesive is able to be elongated by more than 500% before failure.

The process for manufacturing the adhesive is known to those skilled in the art and consists in mixing the various constituents hot, until a homogenous blend is obtained, in a manner similar to the process described, for example, in document EP 912 645 for making strips of adhesive intended for fastening together objects which can be separated by pulling on the tape parallel to the mating plane. The adhesive is extruded or coated so as to obtain a thick sheet. This sheet must have a thickness sufficient to be able to be gripped and to be separated from the prosthesis without breaking, but small enough for the adhesive patches obtained to remain very flexible and conformable and not to modify the apparent volume of the prosthesis. These various conditions are met when the strip of adhesive has a thickness of between 0.6 and 3 mm, and preferably between 0.8 and 2 mm. The coated or extruded sheet of adhesive is then cut by means of a blanking die or a blade so as to obtain strip fragments whose shape matches a portion of the contour of the prosthesis. Finally, these fragments or patches are packaged between two sheets of non-stick paper.

In the case of the use for fastening a breast prosthesis, one of the non-stick protective layers is removed so as to bond the patches to the inner face of the prosthesis which has to be in contact with the skin. The second protective layer is then removed and the prosthesis can be fastened to the skin. Given the choice of constituents, the adhesive is well tolerated and the prosthesis can be removed simply by pulling gently on it, the adhesive sheet remaining attached to the prosthesis without leaving any traces on the skin. The prosthesis, thus adhesively coated, can be kept between two uses by replacing the non-stick protective layer after having cleaned the adhesive, which is hydrophobic in nature, by gently brushing it with water and soap. This cleaning operation makes it possible to remove the skin desquamation residues and consequently to maintain the adhesive properties of the prosthesis, which can thus be repositioned many times. When the adhesion decreases, i.e. after about 3 weeks to 1 month of use, the adhesive patches can be detached from the prosthesis by pulling on one end, parallel to the surface of the prosthesis. The adhesive stretches beyond its yield point without breaking, and is gradually pulled off without leaving any traces of adhesive.

The spent adhesive is thrown away and replaced on the prosthesis with fresh patches which are used in the same way.

Another advantage of this fastening method is the possibility of modifying the position of the points of fastening of the prosthesis; thus, should an area of the skin become irritated or sensitised, the position of the adhesive parts of the prosthesis can very easily be modified so that the sensitive areas of the skin are no longer in contact with the adhesive areas of the prosthesis. This possibility does not exist when the adhesive is inseparable from the prosthesis.

As an example, adhesive patches of oblong shape, having an area of about 20 cm² and a thickness of about 1.3 mm, were produced by extruding a hot-melt adhesive formulated with the following constituents, in the proportions indicated below:

| | | |
|---|---|---|
| Septon 4055 | 9 | parts |
| Kraton G 1726 | 91 | parts |
| Ondina 15 | 75 | parts |
| Escorez 5300 | 280.6 | parts |
| Perkacit ZDBC | 1 | part |
| Irganox 1010 | 1 | part |
| Dye | 1.2 | parts. |

(Septon 4055 is an SEPS triblock elastomer having a high molecular weight, sold by Kuraray; Kraton G 1726 is an SEBS triblock elastomer having a low molecular weight, containing a proportion of diblocks, sold by Shell; Ondina 15 is a liquid paraffin sold by Shell; Escorez 5300 is a C₅ cyclized aliphatic resin sold by Exxon Chemicals; Perkacit ZDBC and Irganox 1010 are antioxidants sold by Akzo and Ciba-Geigy, respectively).

The adhesive is made in a mixer at 160-170° C. and then extruded so as to form a sheet about 1.3 mm in thickness, from which, after cooling, the patches are cut out.

The sheet of adhesive thus obtained has an adhesion of greater than 25N/cm to a polyurethane sheet if the sheet is fastened to an inextensible polyester film and the measurement is carried out by pulling at 90°. The stripping force (that is to say the force to separate the adhesive by pulling on that end of the adhesive sheet which is bonded to a polyurethane sheet in a direction almost parallel to the surface of the latter) is about 1 to 1.3N/cm. The elongation at break of such a sheet is about 965%, with a tensile strength of 3.35N/cm.

The patches thus obtained were used to fasten an external breast prosthesis. The prosthesis was held in place without failing for 25 to 30 consecutive days (it being washed with soapy water each day). No allergy to the adhesive was noted and removing the prosthesis was deemed to be not painful. After being used for one month, the adhesive patches were removed from the prosthesis by pulling the adhesive slowly, parallel to the surface, without tearing the envelope of the prosthesis.

This example must not be regarded as being limiting, and different formulations could be used without departing from the scope of the invention. Likewise, this fastening method may apply to the fastening of other flexible external prosthesis, and to medical accessories which have to remain fastened to the skin, such as, for example, a wig, a stoma pouch, a urine condom or personal monitoring devices used when the wearer is walking about.

1. Method of fastening an external prosthesis or a medical device to the skin by means of a pressure-sensitive adhesive of the type in which at least one cohesive and elastic pressure-sensitive adhesive patch which can be repositioned on the skin is used, and characterized in that the said patch can be detached from the prosthesis by pulling parallel to the surface of the prosthesis.

2. Method according to claim 1, characterized in that the external prosthesis is a flexible and conformable prosthesis, such as a breast prosthesis.

3. Sheet of thermoplastic pressure-sensitive adhesive, for implementing the method according to claim 1, characterized in that it comprises:
1 to 4%, preferably 1 to 2%, of an ABA triblock copolymer having a saturated central block and a molecular weight greater than 180 000;
7 to 20%, preferably 12 to 20%, of an ABA triblock copolymer having a saturated central block and a molecular weight of less than 180 000, the said copolymer possibly containing AB diblock components;
10 to 30% of an oily plasticizer;
55 to 65% of a saturated aliphatic resin whose melting point is above 70° C.;
0.2 to 4% of additives chosen from anti-oxidants, stabilizers, dyes and antibacterial agents.

4. Sheet of adhesive according to claim 3, characterized in that it has a thickness of between 0.6 and 3 mm, sufficient for the cohesive force of the sheet to be greater than the force to remove it from a substrate by pulling parallel to the substrate.

5. Sheet of adhesive according to claim 4, characterized in that the force for pulling off the sheet is about 1 to 1.3N/cm.

6. Sheet of adhesive according to claim 4 or 5, characterized in that the adhesion is greater than 25N/cm.

7. Self-adhesive flexible external prosthesis, characterized in that the adhesive consists of at least one patch of highly cohesive hot-melt adhesive which can be repositioned on the skin and can be detached from the prosthesis by pulling the said sheet parallel to the adhesive-coated surface.
